SEQUENCE LISTING (1 sequence)

SEQ ID NO: 1 - RNA, 21 nt, Artificial Sequence
  Small interfering RNA used to block the expression of thymosin beta-10
aagcggagugaaauuuccuaa

(19) United States
(12) Patent Application Publication
(10) Pub. No.: US 2006/0281678 A1
(43) Pub. Date: Dec. 14, 2006

(54) Novel inhibitor of angiogenesis, tumor progression, and metastasis targeting ras signalling pathway

(76) Inventors: Je-Ho Lee, Seoul (KR); Seung-Hoo Lee, Seoul (KR)

Correspondence Address: SHERIDAN ROSS;SUITE 3500, 1700 LINCOLN STREET, DENVER, CO, 80203, US

(21) Appl. No.: 11/321,186

(22) Filed: Dec. 29, 2005

(30) Foreign Application Priority Data
Jun. 14, 2005 (KR) 10-2005-0051068

(51) Int. Cl. A61K 38/17; A61K 48/00

(52) U.S. Cl. 514/12; 514/44

(57) ABSTRACT

The present invention relates to a novel use of thymosin β₁₀ protein for anti-angiogenesis, anti-tumor progression, and anti-metastasis by targeting a Ras signaling pathway. Particularly, thymosin β₁₀ protein reduces the generation of a Ras-GTP/Raf complex by sequestering Ras-GTP or by interfering with the binding of Ras-GTP to Raf through a direct interaction with Ras, resulting in the inhibition of autocrine and paracrine production of VEGF. Therefore, thymosin β₁₀ protein of the present invention can be effectively used to treat disease conditions associated with angiogenesis, tumor progression, and/or metastasis, particularly those caused by the activation of the Ras signaling pathway, such as solid tumors, rheumatoid arthritis, psoriasis, and diabetic retinopathy.

### CROSS-REFERENCE TO RELATED APPLICATION

This application claims priority to and the benefit of Korean Patent Application No. 10-2005-51068 filed in the Korean Industrial Property Office on Jun. 14, 2005, the contents of which are incorporated herein by reference.

### FIELD OF THE INVENTION

The present invention relates to methods and compositions for modulating angiogenesis, tumor progression, and metastasis of tissues using thymosin β₁₀ or nucleic acids encoding the same. The invention also relates to methods and compositions for modulating Ras activity using thymosin β₁₀ or nucleic acids encoding the same.

### BACKGROUND OF THE INVENTION

Angiogenesis is the process of generating new blood vessels. It plays an important role in neonatal growth of tissues and is believed to be tightly associated with tumor progression and metastasis (Risau W., Nature (1997) 386:671-4). The process is usually mediated by two pathways: generation of new blood vessels by stimulus of preexisting blood vessels, and by a de novo pathway that generates new blood vessels by the division and differentiation of angioblasts, the blood vessel progenitor cells (Blood et al., Bioch. Biophys. Acta (1990) 1032:89-118).

Angiogenesis is generally absent in adults and normal mature tissues except for some cases, such as menstruation and wound healing where neonatal growth of tissues is required, since angiogenesis is highly regulated by a balance between pro-angiogenic factors and anti-angiogenic factors. A disruption of the balance may lead to the onset of a pathological condition generally referred to as angiogenic disease (Folkman et al., Science (1987) 235:442447; Hanahan and Folkman, Cell (1996) 86:353-364; Fidler and Ellis (1994) Cell 79 (2): 185-188). Examples of angiogenic disease include solid tumor growth and metastasis, psoriasis, endometriosis, ischemic disease (e.g., atherosclerosis), chronic inflammatory diseases (e.g., rheumatoid arthritis), some types of solid malignant eye tumors, and diabetic retinopathy. It is well-known that growth and metastasis of solid tumors are angiogenesis-dependent (Folkman, J. Cancer Research (1986) 46: 467-473 and Folkman, J. Journal of the National Cancer Institute (1989) 82: 4-6). The tumors obtain their own blood supply by inducing the growth of new capillary blood vessels. Once these new blood vessels become embedded in the tumor, they provide a means for tumor cells to enter the circulation and metastasize to distant sites, such as the liver, lung, or bone (Weidner, N., et al. The New England Journal of Medicine (1991) 324(1):1-8).

Thus, the control of angiogenesis has been an attractive target for the development of anti-cancer or anti-inflammatory therapy. It has been shown that angiogenesis is controlled by several angiogenic factors such as fibroblast growth factor (FGF), platelet-derived growth factor (PDGF), transforming growth factor alpha (TGFα), hepatocyte growth factor (HGF), and vascular endothelial growth factor (VEGF) (Folkman et al., J. Biol. Chem. (1992) 267:10931-10934).

Among these factors, VEGF is believed to be the primary regulator of angiogenesis (Ferrara N., Nature Review Cancer (2002) 12:795-803). It has been shown that VEGF is upregulated in protein level at sites of pathological angiogenesis and mutant Ras can lead to a marked induction of VEGF (Rak J. et al., Cancer Res. (2000) 60:490-498), suggesting that Ras is involved in angiogenesis (Meadows K N et al., J. Biol Chem (2001) 276:49289-49298).

Ras is a 21 kDa protein that is activated by binding to guanine nucleotides, and functions as a molecular switch that regulates the intracellular GTPase cycle. It has been shown that a Ras signaling pathway is involved in the regulation of a variety of cellular processes from cell growth to cell differentiation by relaying signals that influence growth from the cell surface to the nucleus (Bourne, H. R., et al., Nature (1991) 349:117-127). The fact that mutated Ras genes are common and are found in more than 30% of human cancers supports its critical role in cell signaling pathway.

The signaling pathway via Ras has therefore attracted considerable attention as a target for anti-angiogenesis therapy.

WO 01/012210 discloses the inhibition of angiogenesis by regulating the activity of Raf, a protein working downstream of Ras in a Ras signaling pathway.

WO 97/16547 discloses the inhibition of Ras expression by using a Ras antisense oligonucleotide.

WO 03/086467 discloses a compound that is used to treat cancer by inhibiting Ras and proteins working downstream of Ras.

WO 03/018755 discloses the regulation of tumor progression by controlling the expression of Ras.

WO 2005/027972 discloses the therapeutic benefit of VEGF inhibitor for treating hyperplastic diseases associated with angiogenesis.

WO 02/28381 discloses the inhibition of Ras activity by regulating the farnesylation of Ras.

Korean patent No. 0454871 discloses thymosin β₁₀ as a therapeutic agent for gene therapy for ovarian cancer.

Even though various therapeutic agents influencing the Ras signaling pathway have been identified, they only show a limited efficacy for the regulation of the Ras signaling pathway and there is still an unmet need for development of a new therapeutic agent targeting Ras. Further, none of the disclosures show or suggest the inhibition of angiogenesis, tumor progression, and/or metastasis using thymosin β₁₀ through a sequestration of Ras-GTP or an inhibition of Ras-GTP binding to Raf by direct interaction with Ras.

The information disclosed in this Background of the Invention section is only for enhancement of understanding of the background of the invention and therefore, unless explicitly described to the contrary, it should not be taken as an acknowledgement or any form of suggestion that this information forms the prior art that is already known in this country to a person of ordinary skill in the art.

### SUMMARY OF THE INVENTION

The present invention has been made in an effort to provide a therapeutic agent that is capable of effectively inhibiting the activity of Ras and the Ras signaling pathway, and the present inventors have discovered that thymosin β₁₀ can effectively inhibit the activity of Ras leading to the inhibition of the Ras signaling pathway thereby inhibiting angiogenesis, tumor progression, and/or metastasis.

The present invention therefore contemplates modulation of angiogenesis, tumor progression, and/or metastasis in tissues by administering to a tissue or a subject associated with a disease condition a therapeutically effective amount of a thymosin β₁₀ or a nucleotide sequence encoding said protein. In one embodiment, such disease conditions are mediated or result from the activity of Ras. In another embodiment, said modulating inhibits angiogenesis, tumor progression, and/or metastasis.

The present invention further contemplates a method of inhibiting Ras activity in a tissue or a subject including the step of administering to a tissue or a subject associated with a disease condition a therapeutically effective amount of a thymosin β₁₀ or a nucleotide sequence encoding said protein.

In one embodiment, the thymosin β₁₀ causes a sequestration of Ras-GTP or an inhibition of Ras-GTP binding to Raf by direct interaction with Ras, leading to a reduction in the expression of VEGF, which is a clear implication for anti-angiogenesis and anti-cancer therapy.

The present invention also contemplates pharmaceutical compositions for inhibiting angiogenesis, tumor progression, and/or metastasis in a target mammalian tissue comprising thymosin β₁₀ or a nucleotide sequence encoding said protein as an active ingredient, and a pharmaceutically acceptable carrier or excipient.

In one embodiment, the cells or tissues to be treated can be any cell or tissue where modulation and particularly inhibition of angiogenesis, tumor progression, and/or metastasis are desirable. Exemplary cells or tissues include inflamed tissue, solid tumors, metastasis, tissues undergoing restenosis, and the like. Hence, the methods and compositions of the present invention are useful for treating disease conditions where deleterious angiogenesis, tumor progression, and/or metastasis are occurring. Such conditions include, for example, solid tumors, including but not limited to colon cancers, ovarian cancers, lung cancers, lymphomas, breast cancers, prostate cancers, lymphomas, and renal cell cancers; rheumatoid arthritis; psoriasis; diabetic retinopathy; diabetic nephropathy; hypertension; endometriosis; and adiposis.

### BRIEF DESCRIPTION OF THE DRAWINGS

FIG. 1 illustrates the thymosin β₁₀-mediated inhibition of VEGF-induced HUVEC tumor progression. The abbreviations are as follows: Un: negative control, uninfected HUVEC, treated or not treated with VEGF; Ad: HUVEC infected with adenovirus not containing thymosin β₁₀; AdT β₁₀: HUVEC infected with adenovirus containing thymosin β₁₀; AdT β₁₀+siRNA: HUVEC treated with AdT β₁₀ and siRNA. “*” and “***” indicate statistical significance of P<0.05 and P<0.001, respectively, compared with Un treated with VEGF.

FIG. 2 depicts a bar graph showing thymosin β₁₀₋mediated inhibition of VEGF-induced HUVEC metastasis (FIG. 2A) and invasion (FIG. 2B), and abbreviations are the same as above. “*”, “**”, and “***” indicate statistical significance of P<0.05, P<0.01, and P<0.001, respectively, compared with untreated with VEGF.

FIG. 3 depicts phase contrast images showing the inhibitory effect of thymosin β₁₀ on tube formation of HUVEC in vitro (FIGS. 3A-3E), and a graph showing the corresponding tube length formed in each sample (FIG. 3F). GFP (Green Fluorescence Protein) images showing the expression of thymosin β₁₀ are shown (bottom picture). The abbreviations are the same as above. The arrows in 3D indicate thin and broken tubes, compared with control (FIGS. 3B and 3C) not treated with thymosin β₁₀.

FIG. 4 depicts phase contrast image (Magnification: ×12.5 (top); ×40 (bottom)) of cross sections of mouse tibialis anterior muscle showing the inhibitory activities of thymosin β₁₀ (FIGS. 4A-4D) and Paclitaxel (FIG. 4E) on vessel sprouting. The arrows indicate the outgrowth of capillary-like structures, and the bar is 500 μM. FIG. 4F depicts a graph showing the mean area of vascular sprouting in each corresponding sample, and the abbreviations are the same as above.

FIG. 5 illustrates the interaction of Ras with either thymosin β₁₀ (FIG. 5A) or thymosin β₄ (FIG. 5B). The interactions were presented by the relative activity of β-galactosidase.

FIG. 6 illustrates the direct interaction of thymosin β₁₀ with Ras in vitro. The GST pull-down assay was done using thymosin β₁₀ fused with GST (GST-Tβ₁₀) and anti-GST antibody. The bound K-Ras was detected with anti-His antibody.

FIG. 7 illustrates that thymosin β₁₀ interferes with VEGF-mediated Ras-ERK signaling and reduces the production of VEGF. FIG. 7A illustrates the effect of thymosin β₁₀ on the formation of active Ras (Ras-GTP) in HUVEC using a GST-Raf-RBD pull-down assay. FIG. 7B is the result of thin layer chromatography (TLC) of nucleotide eluted from immunoprecipitates of Ras from HUVEC, and the quantification of the signals is shown in FIG. 7C. FIG. 7D is the result of western blot analysis showing that GFP-T β₁₀, Ras, and alpha-tubulin are located in cytoplasm, nuclear membrane, and nucleus, respectively. FIG. 7E illustrates the effect of over expression of thymosin β₁₀ in HUVECs on the activation of MEK and ERK proteins, which function in a Ras signaling pathway downstream of Ras. FIGS. 7E and 7F illustrate the effect of thymosin β₁₀ on the VEGF production in HUVEC (FIG. 7E) and 2774 ovarian cancer cell line (FIG. 7F). Secreted VEGF was detected in the concentrated conditioned medium (CCM) in FIG. 7F. The numbers in FIG. 7D are as follows: 1: HUVEC neither treated with VEGF nor infected with adenovirus; 2: HUVEC treated with VEGF but infected with adenovirus not containing thymosin β₁₀ (Ad); 3: HUVEC both treated with VEGF and infected with adenovirus containing thymosin β₁₀.

FIG. 8 illustrates the inhibitory effect of thymosin β₁₀ (AdT β₁₀) on tumor growth and angiogenesis in an s.c. tumor model. FIGS. 8A and 8B show the direct comparison of tumor volume in mice infected with adenovirus either containing thymosin β₁₀ (AdT β₁₀) or adenovirus alone (Ad). FIG. 8C shows cross sections of the tumors stained using anti-CD31 antibody specific for vascular endothelial cells and FIG. 8D shows the number of blood vessels per tumor section.

FIG. 9 illustrates the inhibitory effect of thymosin β₁₀ on tumor growth and angiogenesis in an orthotopic tumor model. FIGS. 9A and 9B show the direct comparison of tumor volume in mice infected with adenovirus either containing thymosin β₁₀ (AdT β₁₀) or adenovirus alone (Ad). Excised tumors (white arrow) and normal ovaries (white arrowhead) from Ad- or AdT β₁₀ treated mice 10 days after virus injection are shown (FIG. 9A bottom). FIG. 9C shows frozen sections of tumors stained using anti-CD31 antibody specific for vascular endothelial cells and FIG. 9D shows the number of blood vessels per tumor section. The bar represents 50 μM, and “*”, “**”, and “***” indicate statistical significance of P<0.05, P<0.01, and P<0.001, respectively, compared with Ad in FIGS. 8 and 9.

### DETAILED DESCRIPTION OF THE PREFERRED EMBODIMENTS

In order that the present invention herein described may be fully understood, the following detailed description is set forth.

Unless otherwise defined, all technical terms used herein have the same meaning as commonly understood by one of ordinary skill in the pertinent art. Commonly understood definitions of molecular biology terms can be found in, for example, Rieger et al., Glossary of Genetics: Classical and Molecular, 5th edition, Springer-Verlag: New York, 1991; and Lewin, Genes VIII, Oxford University Press: New York, 2004.

The present invention is based on the discovery of novel use of thymosin β₁₀ for inhibition of angiogenesis, tumor progression, and/or metastasis through direct interaction with Ras. In one aspect, the present invention therefore provides methods and compositions for modulating angiogenesis, tumor progression, and/or metastasis of tissues using thymosin β₁₀ or nucleic acids encoding the same through its interaction with Ras. In another aspect, the present invention provides methods and compositions for modulating Ras activity using thymosin β₁₀ or nucleic acids encoding the same.

Thymosin β₁₀ belongs to a family of thymosin β proteins, whose sequence is well conserved among different species and is found in most mammalian species. It is known that thymosin β₁₀ inhibits barbed end actin polymerization by sequestering actin monomers resulting in the depolymerization of the intracellular F-actin network (Nachmias, Curr. Opin. Cell Biol. (1995) 5:56-62; Yu et al., J. Biol. Chem. (1993) 268:502-509; Yu et al., Cell Motil. Cytoskeleton (1994)27:13-25). Among the members of the thymosin β family, thymosin β₄ and β₁₀ are the two most widely expressed ones in mammalian cells, and in particular, thymosin β₁₀ are expressed in tissues of the pancreas, thymus, prostate, testis, and colon. It is also known that thymosin β₁₀ is highly expressed during development (Carpintero et al., FEBS Left. (1996) 394:103-6) and is also involved in cell detachment (Iguchi et al., Eur. J. Biochem. (1998) 253:766-770). However, the involvement of thymosin β₁₀ in the Ras signaling pathway has not been previously known.

Thymosin β₁₀ of the methods and the compositions of the present invention encompass purified protein, biologically active protein fragments, recombinantly produced thymosin β₁₀ or fragments thereof or fusion proteins, or gene/nucleic acid expression vectors for expressing thymosin β₁₀, or any functional variant thereof, all of which can be prepared by methods that are well-known in the art.

Further, thymosin β₁₀ of the present invention or a gene encoding the same may be selected from known sequences of various origins including, but not limited to, human and rat. For example, thymosin β₁₀ protein sequence with GeneBank Accession No. P63313 for human and P63312 for rat, and a gene encoding the same with GeneBank Accession No. M92381 for human and M17698 for rat may be used.

As used herein, the term “gene” is well-known in the art and refers to a nucleic acid sequence defining a single protein or polypeptide. It will be readily recognized by those of ordinary skill that the nucleic acid sequence of the present invention can be incorporated into any one of numerous kits which are well-known in the art.

As used herein, the term “functional variant” refers to a protein or nucleic acid molecule which is substantially similar in biological activity to the protein or nucleic acid of the present invention. Such functional variant encompasses, for example, base insertions, deletions, or substitution mutants that may be generated spontaneously or artificially by methods that are well-known in the art, e.g., by primer-directed PCR (Kramer, W. & Fritz, H J. Methods in Enzymology (1987) 154:350-367), “error-prone” PCR (Cadwell, R. C. and G. F. Joyce, PCR methods Appl (1992) 2:28-33), “gene-shuffling” called PCR-reassembly of overlapping DNA fragments, and the like. Also encompassed by the present invention is the mutation at the nucleic acid level that does not change an amino acid such as a degenerate variant due to the degeneracy of the genetic code.

A gene encoding thymosin β₁₀ protein of the present invention encompasses genomic DNA, cDNA, and synthetic or recombinantly produced DNA, all of which can be prepared by methods that are well-known in the art. For example, genomic DNA is extracted from cells expressing thymosin β₁₀, which is subsequently used for the construction of a genomic library using vectors such as plasmid, phage, cosmid, BAC, and PAC followed by colony hybridization or plaque hybridization depending on the vectors to screen the thymosin β₁₀ genomic DNA using a probe with a sequence specific for thymosin β₁₀ of the present invention. For the preparation of cDNA, mRNA extracted from cells expressing thymosin β₁₀ is used to synthesize first strand cDNA by reverse-transcription followed by PCR for the amplification of thymosin β₁₀ cDNA using the primers specific for thymosin β₁₀ of the present invention.

Preferably, such functional variant or isolated sequence-fragment is at least 50%, more preferably 70%, and most preferably 90% homologous to the corresponding part of the natural human or rat thymosin β₁₀ gene.

In one embodiment, the present invention provides methods and compositions for inhibiting angiogenesis, tumor progression, and/or metastasis by providing thymosin β₁₀ or a nucleotide sequence encoding said protein. Thymosin β₁₀ of the present invention can effectively inhibit angiogenesis, tumor progression, and/or metastasis by suppressing the Ras signaling pathway through direct interaction with Ras.

Thymosin β₁₀ of the present invention exerts effects through its direct interaction with Ras, and Ras that interacts with thymosin β₁₀ of the present invention includes H-Ras, K-Ras, and N-Ras found in cells (Sharpe C et al., J Am Soc Nephrol (2000) 11:1600-1606). In one embodiment, it is K-Ras that interacts with thymosin β₁₀ of the present invention. K-Ras accounts for more than 95% of total Ras expressed in cells. The difference in functions among those isoforms is not yet known. The wild type DNA sequences of N—, K—, and H-Ras are provided as GenBank Accession No. AF493919, AF493916 and M54968, respectively.

Further, Ras protein that interacts with thymosin β₁₀ of the present invention may be active GTP-bound Ras or inactive GDP-bound Ras, and particularly active Ras bound to GTP. Furthermore, Ras protein that interacts with thymosin β₁₀ of the present invention may include wild type or mutant Ras. The mutant Ras includes one with well-known amino acid substitution at positions 12, 13, or 61, for instance, one with glycine at position 12 being replaced with valine. In one embodiment, thymosin β₁₀ of the present invention directly interacts with Ras-GTP.

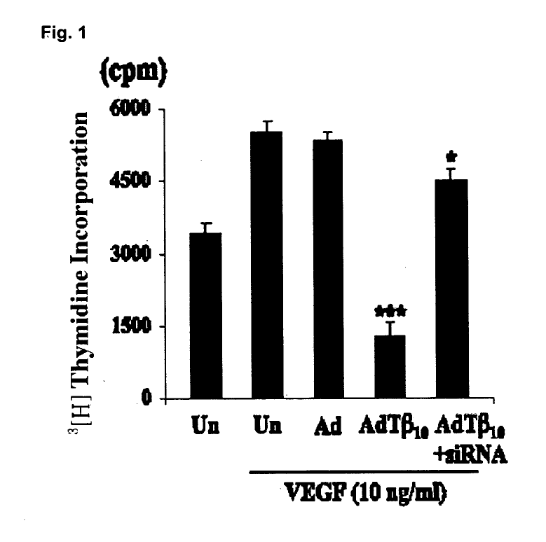

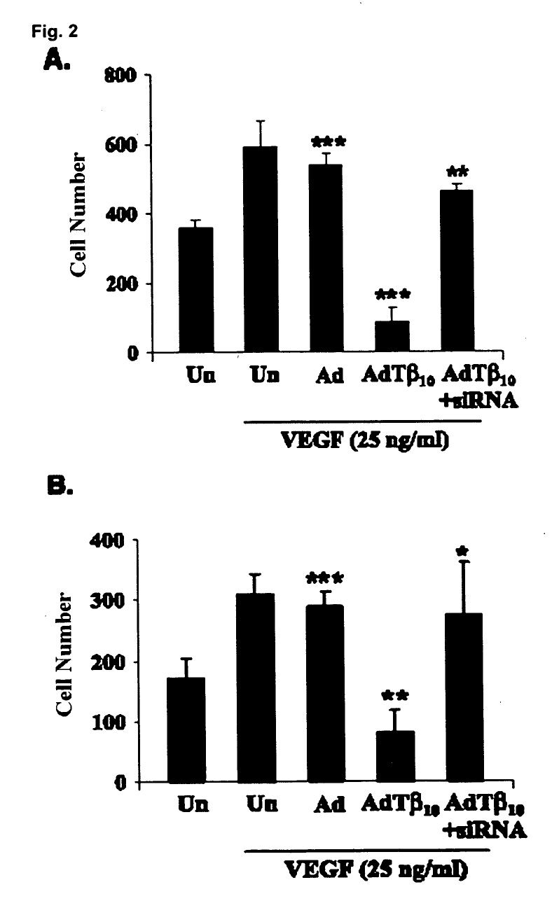

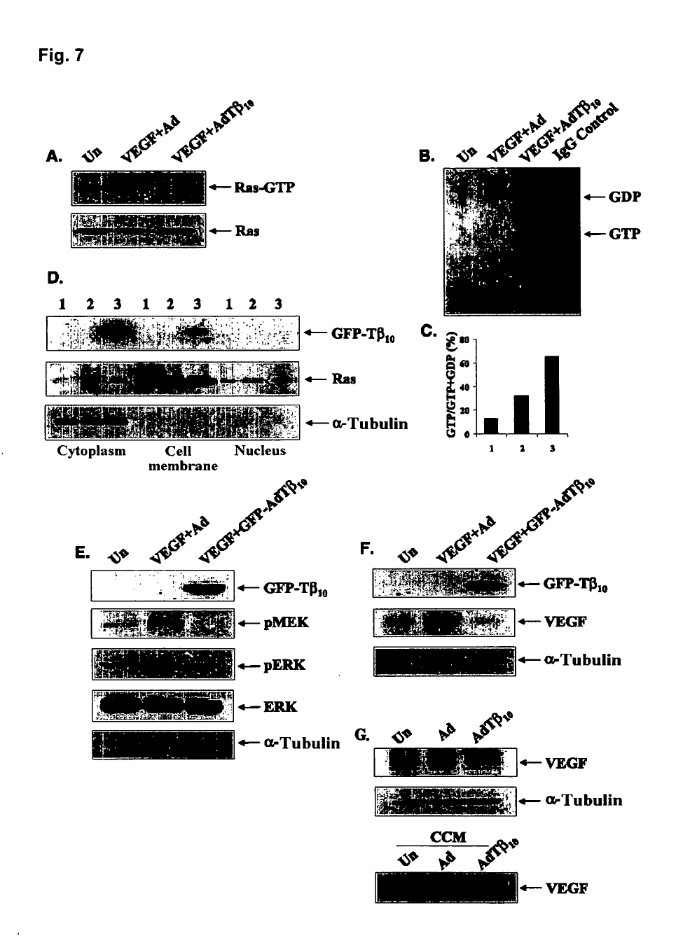

According to one embodiment, the direct interaction of thymosin β₁₀ of the present invention with Ras causes a sequestration of Ras-GTP and/or an inhibition of the binding of Ras-GTP to Raf. In a further embodiment, the over expression of thymosin β₁₀ of the present invention cell significantly decreases the expression and secretion of VEGF both in endothelial cells and in tumor cells (see FIGS. 7F and 7G) suggesting that thymosin β₁₀ not only inhibits the autocrine VEGF in endothelial cells but also inhibits paracrine induction of VEGF in tumor cells, and it further inhibits VEGF-mediated tumor progression, metastasis, and cell invasion (see FIG. 1 and FIG. 2), indicating that thymosin β₁₀ has an inhibitory effect on angiogenesis, tumor progression, and metastasis.

According to another embodiment, the level of Ras-GTP/Raf complex of the Ras signaling pathway negatively affects the pathway downstream of Ras leading to Raf, MAPKKK (MAPK kinase kinase), MAPKK (MAPK kinase or MEK) and MAPK (Mitogen-Activated Protein Kinase, also known as Extracellular signal Regulated Protein Kinase (ERK)) (Neiman, A., TIGS (1993) 9(II): 390-394) activation, which is often activated in cancer cells (Gu, Z., et al., J. Virol. (1995) 69(12):8051-8056). ERK further activates target molecules such as VEGF.

VEGF has a very specific activity for promoting cell division, especially of endothelial cells, and the expression of VEGF in endothelial cells is essential for angiogenesis and vasculogenesis in adults (Carmeliet et al., Nature (1996) 380:435439).

Thus, taken together, the reduction in the production of VEGF after treating the cell with thymosin β₁₀ clearly indicates the efficacy of the thymosin β₁₀ of the present invention as a therapeutic agent for angiogenesis, tumor progression, and metastasis.

According to further embodiment, sub-cellular localization of thymosin β₁₀ both in a cell membrane and cytoplasm suggest its interaction with Ras which is normally present in the cell membrane, and its inhibition of binding of Ras-GTP to Raf which normally occurs in the cytoplasm.

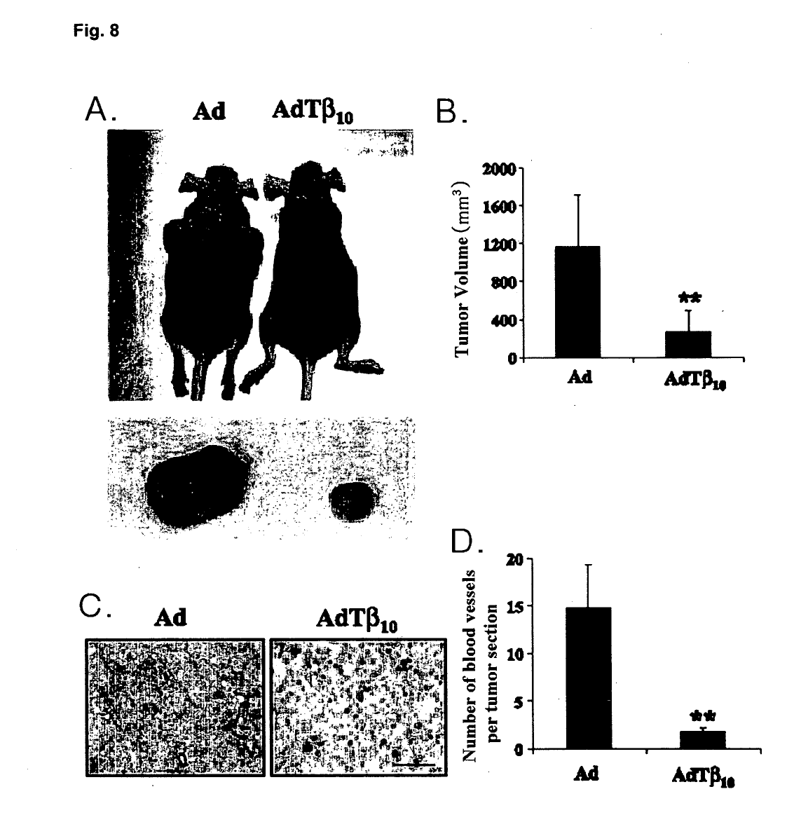

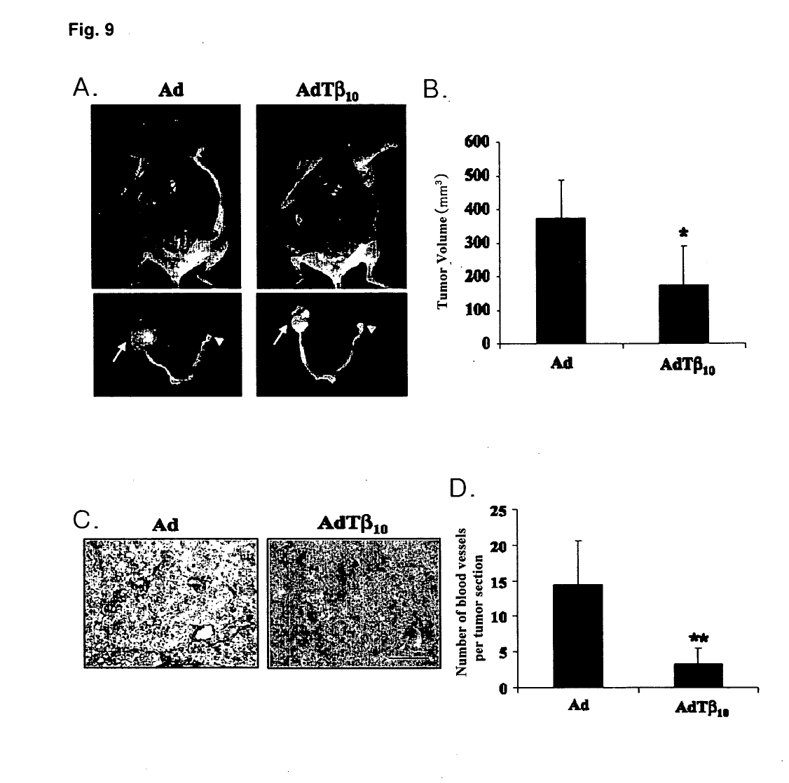

The inhibitory effect of thymosin β₁₀ on angiogenesis and tumor progression has been confirmed in vivo. In one embodiment, using a heterotopic and orthotopic animal model, tumor progression and angiogenesis were significantly inhibited, compared with a negative control (see FIG. 8 and FIG. 9) while neither weight loss nor hepatotoxicity were observed, indicating the absence of toxicity and thereby enabling the safe use of thymosin β₁₀ of the present invention for the treatment of disease conditions associated with angiogenesis, tumor progression, and metastasis.

The methods and compositions providing thymosin β₁₀ of the present invention can therefore be used to treat the tissues associated with the disease condition where inhibition of angiogenesis, tumor progression, and/or metastasis are desirable. In one embodiment, such disease conditions depend on the activity of Ras.

The tissues to be treated can be any tissue where modulation, particularly inhibition, of angiogenesis, tumor progression, and/or metastasis are desirable. Exemplary cells or tissues include inflamed tissue, solid tumors, metastasis, tissues undergoing restenosis, and the like. Hence, the methods and compositions of the present invention are useful for treating disease conditions where deleterious angiogenesis, tumor progression, and/or metastasis are occurring. Such conditions include, for example, solid tumors, including but not limited to colon cancers, ovarian cancers, lung cancers, lymphomas, breast cancers, prostate cancers, lymphomas, renal cell cancers; rheumatoid arthritis; psoriasis; diabetic retinopathy; diabetic nephropathy; hypertension; chronic hepatitis; endometriosis; and adiposis (Folkman et al., Science (1987) 235:442-447; Hanahan and Folkman, Cell (1996) 86:353-364; Fidler and Ellis Cell (1994) 79(2): 185-188; Sparmann et al., Cancer Cell (2004) 6(5) :447-458; List, Oncologist (2002)7 Suppl 1:39-49, 2002; Griffioen and Molema, Pharmacological Reviews (2001) 52: 237-268).

Specifically, for solid tumors characterized by uncontrolled tumor progression and metastasis, for which angiogenesis is required (Hanahan, D. et al., Cell (1996) 86:353-364), the inhibitor of the present invention containing thymosin β₁₀ or the gene encoding the same can be effectively used for the treatment of all types of solid tumors. In one embodiment of the present invention, the ectopic expression of thymosin β₁₀ of the present invention was able to effectively suppress ovarian cancer (see Example 9).

The involvement of angiogenesis in diseases such as Diabetic retinopathy (Barinaga, N., Science (1995) 267:452-453; Noma H. et al., Arch Ophthalmol (2002) 120(8):1075-1080), rheumatoid arthritis (Paleolog E M et al., Springer Semin Immunopathol (1998) 20(1-2):73-94; Koch, Ann Rheum Dis (2000) 59 (Suppl 1):165-171) and-other inflammatory related diseases are well-known in the art, and it will be readily recognized by those of ordinary skill that thymosin β₁₀ of the present invention can be used to treat such disease conditions.

For the expression of thymosin β₁₀ provided for the methods and compositions of the present invention, a thymosin β₁₀ gene or functional variant thereof as described herein operatively link to a vector. The choice of said vector depends, as is well-known in the art, on protein expression, the host cell to be transfected, and the like. A vector contemplated by the present invention is at least capable of directing the replication and expression of a gene included in the vector in cells, preferably in eukaryotic cells.

Such eukaryotic expression vectors encompass both viral and non-viral vectors, and are familiar to one of ordinary skill in the pertinent art. For non-viral vector systems, see for examples of Ausebel, et al., in Current Protocols in Molecular Biology, Wiley and Sons, New York (1993) and of Sambrook et al, Molecular Cloning: A Laboratory Manual, Cold Spring Harbor Laboratory (1989). Also they are commercially available from several sources. Typical of such vectors are pCDNA 3 or 4, pRc/CMV (Invitrogen, Carlsbad, Calif., USA), pSVL, and pKSV-10 (Amersham Pharmacia Biotech, Piscataway, N.J., USA).

The viral expression vectors for the expression of thymosin β₁₀ include infectious vectors such as recombinant DNA viruses, and adenovirus or retrovirus vectors which are engineered to express the desired protein and have features that allow infection of target tissues, for example such viral vectors are used encapsulated by a viral coat, which is familiar to one of ordinary skill in the pertinent art (see Logan et al., Proc. Natl. Acad. Sci., USA (1984) 81:3655-3659; Mackett et al., Proc. Natl. Acad. Sci., USA (1982) 79:4927-4931; Cone et al., Proc. Natl. Acad. Sci., USA (1984) 81:6349-6353). Further, retroviral/adenoviral expression systems can be readily adapted for practice of the methods and compositions of the present invention. For example, see Karavanas et al., Crit. Rev. in Oncology/Hematology (1998) 28:7-30 for retroviral viral vectors, and Gene Expression Systems ed., Fernandez and Hoeffler, Academic Press, San Diego, USA, 1990, for adenoviral expression systems. In one embodiment, thymosin β₁₀ is expressed using adenoviral expression systems such as described in Korean Patent No. 454871, and Lee et al., Cancer Res (2005) 65:137-147.

In one aspect, the present invention provides a method for inhibiting angiogenesis, tumor progression, and metastasis in a tissue and a subject with a disease process or condition, where such disease process and conditions are mediated or resulting from the activity of Ras. The method comprises administering to said tissue or subject a therapeutically effective amount of thymosin β₁₀ or a nucleotide sequence encoding said protein.

As used herein, the term “therapeutically effective” is an amount of thymosin β₁₀ or a nucleotide sequence encoding said protein sufficient to produce a measurable modulation, preferably inhibition, of angiogenesis, tumor progression, and metastasis in tissue or a subject. Modulation of angiogenesis, tumor progression, and metastasis is measured by methods known to one skilled in the art, for example, those described in the examples.

The tissue to be treated may be any of a variety of tissues, or organs including skin, muscle, gut, connective tissue, brain tissue, bones, and the like in which blood vessels can invade upon angiogenic stimuli and/or from which a tumor can arise.

The subject is a patient to be treated, wherein the patient is a human as well as a veterinary patient.

The pharmaceutical composition containing thymosin β₁₀ or a nucleotide sequence encoding said protein of the present invention is administered in a manner compatible with the dosage formulation, and in a therapeutically effective amount, for example intravenously, intraperitoneally, intramuscularly, subcutaneously, and intradermally. It may also be administered by any of the other numerous techniques known to those of skill in the art, see for example the latest edition of Remington's Pharmaceutical Science, which is incorporated herein by reference.

For example, for injections, the thymosin β₁₀ or a nucleotide sequence encoding said protein of the invention may be formulated in adequate solutions including but not limited to physiologically compatible buffers such as Hink's solution, Ringer's solution, or a physiological saline buffer. The solutions may contain formulatory agents such as suspending, stabilizing, and/or dispersing agents. Alternatively, thymosin β₁₀ or a nucleotide sequence encoding said protein of the invention may be in powder form for combination with a suitable vehicle, e.g., sterile pyrogen-free water, before use.

Further, the composition of the present invention may be administered per se or may be applied as an appropriate formulation together with pharmaceutically acceptable carriers, diluents, or excipients that are well-known in the art.

In addition, other pharmaceutical delivery systems such as liposomes and emulsions that are well-known in the art, and a sustained-release system, such as semi-permeable matrices of solid polymers containing the therapeutic agent may be employed. Various sustained-release materials have been established and are well-known to one skilled in the art.

Further, the composition of the present invention can be administered alone or together with another therapy conventionally used for the treatment of angiogenesis, tumor progression, and/or metastasis related diseases, such as surgical operation, hormone therapy, chemotherapy, or biological agents.

The quantity to be administered and timing may vary within a range depending on the formulation, the route of administration, and the tissue or subject to be treated, e.g., the patient's age, body weight, overall health, and other factors. For the nucleic acid sequence, the amount administered depends on the properties of the expression vector, the tissue to be treated, and the like. The suitable amount can be measured by amount of vector used, or amount of expressed protein expected. The exact formulation, route of administration, and dose can be chosen by the individual physician in view of the patient's condition (see, for dose and dosing schedule, e.g. latest editions of Remington's Pharmaceutical Science, Mark Publishing Co., Easton, Pa.; and Goodman and Gilman's: The Pharmacological Basis of Therapeutics, Pergamon Press).

The dosage of the thymosin β₁₀ or a nucleotide sequence encoding said protein of the invention preferably lies within a range of concentrations that include the effective dose with little or no toxicity, but that are sufficient to produce a measurable modulation, preferably inhibition, of angiogenesis, tumor progression, and metastasis in tissue or a subject. A single dose of the thymosin β₁₀ or a nucleotide sequence encoding said protein administered will typically be in the range of about 0.05 to about 10 mg/kg of patient weight. The thymosin β₁₀ or a nucleotide sequence encoding said protein of the invention will typically be formulated in a suitable formulation at concentrations of about 0.001 mg/ml to 100 mg/ml such that the final dose is about 0.05 to 10 mg/kg of patient body weight. For viral vectors, the recombinant virus containing such viral vectors will typically be in the range of about 10³˜10¹² pfu/kg per kg of body weight.

The following examples illustrate the present invention in further detail. However, it is understood that the present invention is not limited by these examples

### EXAMPLE 1

### Construction of Expression Vectors and siRNA Targeting Thymosin β₁₀

Various viral and non-viral vectors expressing thymosin β₁₀, β₄, or K-Ras were constructed and used to investigate the inhibitory effect of thymosin β₁₀ of the present invention on angiogenesis, tumor progression, and metastasis.

For viral constructs, adenoviral expression systems were used, and adenoviral vectors GFP-AdTβ₁₀, AdTβ₁₀, and AdTβ₄, each expressing thymosin β₁₀ fused to Green Fluorescence Protein (GFP), thymosin β₁₀, and thymosin β₄, respectively, were prepared as described (Lee et al., Oncogene (2001) 20:6700-6776; Lee et al., Cancer Res (2005) 65:137-147; Korean Patent No. 454871).

For non-viral constructs, vectors such as pGEX4T-1 (Amersham Pharmacia Biotech) and pcDNA4HisMax (Invitrogen) that are capable of expressing a protein fused to GST (glutathione S transferase) and 6 His-residues, respectively, were used to construct expression vectors pGST-Tβ₁₀ and pGST-Tβ₄, and pHis-Ras as described (Lee et al., Oncogene (2001) 20:6700-6776; Lee et al., Cancer Res (2005) 65:137-147; Korean Patent No. 454871).

Proteins fused to GST or 6 histidine residues are useful for the isolation and purification of a protein as well as for analysis of the interaction between the proteins.

siRNA (small interfering RNA) was used to block the expression of thymosin β₁₀. The siRNA targeting thymosin β₁₀ (AAGCGGAGUGAAAUUUCCUAA) correspond to nucleotides 199 to 217 of the human sequence was synthesized using an siRNA construction kit (Ambion, Austin, Tex., USA) according to the manufacturer's instructions, and transfected into cells by using the RNAi shuttle (Orbigen, San Diego, Calif., USA) according to the manufacturer's instructions as previously described (Lee et al., Oncogene (2001) 20:6700-6776; Lee et al., Cancer Res (2005) 65:137-147).

### EXAMPLE 2

### Cell Culture and Transfection

HUVEC (human umbilical vein endothelial cell, Clonetics, San Diego, Calif., USA), ovarian cancer cell line 2774 (Clonetics), and 293 (Clonetics) cells were cultured as described (Lee et al., Oncogene (2001) 20:6700-6776; Lee et al., Cancer Res (2005) 65:137-147). Briefly, HUVECs were grown on a plate coated with 0.3% gelatin using an EGM-2 kit (Clonetics). The ovarian cancer cell line 2774 (Clonetics) and 293 cells (Clonetics) for the preparation of adenovirus were cultured in DMEM (Dulbecco's Modification of Eagles Medium) and EMEM (Eagles Minimum Essential Medium), respectively, each supplemented with 10% FBS and antibiotics (Life Technologies, Gaithersburg, Md., USA). The cells were incubated at 37□, with 5% CO₂ in an air atmosphere.

DNA transfection into the cells and adenovirus infection were carried out as described (Lee et al., Oncogene (2001) 20:6700-6776; Lee et al., Cancer Res (2005) 65:137-147).

### EXAMPLE 3

### The Inhibitory Effect of Thymosin β₁₀ on Cell Proliferation

To investigate the effect of thymosin β₁₀ on proliferation of endothelial cells, which are essential for angiogenesis, HUVEC cells were infected with adenovirus containing an empty vector not expressing thymosin β₁₀ (Ad), adenovirus expressing thymosin β₁₀ (AdTβ₁₀) or AdTβ₁₀, followed by transfection with siRNA targeting thymosin β₁₀ as described in Example 1 (AdTβ₁₀+siRNA) as described (Lee et al., Oncogene (2001) 20:6700-6776; Lee et al., Cancer Res (2005) 65:137-147).

Briefly, 18 hours after the infection, each of the cells was treated with 10 ng/ml of VEGF (R&D systems, Minneapolis, Minn., USA) for 24 hours to stimulate tumor progression, and the tumor progression in each experimental group was measured by a [³H] methylthymidine incorporation assay. [³H] methylthymidine (0.5 μCi/ml, Amersham Pharmacia Biotech) was added to the cells 4 hours prior to the assay. Then, the cpm (count per minute) of thymidine incorporated into the genomic DNA of the cell was measured with a liquid scintillation counter (Beckman, Fullerton, Calif.). The experiments were carried out in triplicate and each cpm value represents the mean±standard deviation (SD) of the triplicate samples.

As shown in FIG. 1, the cells were successfully induced to proliferate by stimulation with VEGF as compared to a negative control not treated with VEGF (Un vs. Un treated with VEGF). However, such proliferation of cells was significantly reduced by the treatment of cells with AdT β₁₀ as indicated by the cpm of HUVEC infected with AdT β₁₀ that was much lower than those of its corresponding negative controls, i.e., cells not infected with the virus at all (Un) and cells infected with Ad. However, such inhibitory effect of thymosin β₁₀ was not observed in HUVEC treated with both AdTβ₁₀ and siRNA which had a proliferation level that was comparable to those of negative controls, clearly indicating that the inhibition of cell proliferation is indeed attributed to thymosin β₁₀. Thus, it has been confirmed that the expression of thymosin β₁₀ can effectively inhibit VEGF-mediated cell proliferation, and thus angiogenesis.

### EXAMPLE 4

### The Inhibitory Effect of Thymosin β₁₀ on Migration and Invasion

To investigate the effect of thymosin so on migration and invasion, transwell migration and invasion assays were carried out (8-μM pore size, Costar, Cambridge, Mass.) as described (Lee et al., Biochem Biophys Res Commun (1999) 264:743-750, Lee et al., Cancer Res (2005) 65:137-147).

Briefly, for the migration assay, the lower surface of a filter was coated with 10 μg of gelatin. Lower wells were filled M199 (Life Technologies) containing 1% FBS with VEGF (25 ng/ml). Upper wells were seeded with uninfected HUVECs and HUVECs treated with each of Ad, AdTβ₁₀, and AdTβ₁₀+siRNA such that the final cell density was 1×10⁴ cells/100 μl for each well, followed by incubation for 24 hours. The cells were then fixed and stained with H&E (Hematoxylin and Eosin) (BioRad, Hercules, Calif., USA). Non-migrating cells remaining on the upper surface of the filter were removed by wiping the cells with a cotton swab. The number of cells that migrated into the lower side of the filter were counted under a light microscope, and mean values of eight fields were calculated.

For the invasion assay, the lower and upper surfaces of a filter were coated with 10 μg of gelatin and 10 μg of Matrigel (BD Biosciences, Bedford, Mass.), respectively. Upper wells were seeded with uninfected HUVECs and HUVECs treated with each of Ad, AdT β₁₀, and AdT β₁₀+siRNA such that the final density of cells was 1×10⁴ cells/100 μl for each well, followed by incubation for 30 hours. The cells were then fixed and stained, and quantified as above.

As shown in FIG. 2, the cells were successfully induced to migrate (FIG. 2A) and invade (FIG. 2B) by stimulation with VEGF as compared to a negative control not treated with VEGF (Un vs. Un treated with VEGF). However, such migration and invasion of cells were significantly reduced by the treatment of cells with AdT β₁₀ as indicated by a much lower number of cells that migrated and invaded for cells infected with AdT β₁₀ than those of its corresponding negative controls, i.e., cells not infected with the virus at all (Un) and cells infected with Ad. However, such inhibitory effect of thymosin β₁₀ was abrogated in HUVEC treated with both AdTβ₁₀ and siRNA resulting in that the number of migrating and invading cells was comparable to those of the negative controls, clearly indicating that such inhibitory effect is indeed attributed to thymosin β₁₀. Thus, these results clearly demonstrate that the expression of thymosin β₁₀ can effectively inhibit VEGF-mediated cell migration and invasion.

### EXAMPLE 5

### The Inhibitory Effect of Thymosin β₁₀ on Tube Formation and ex vivo Angiogenesis

In order to confirm the anti-angiogenesis property of thymosin β₁₀, the effect of thymosin β₁₀ on tube formation and angiogenesis was investigated as follows.

### EXAMPLE 5-1

### The Inhibitory Effect of Thymosin β₁₀ on Tube Formation

The inhibitory effect of thymosin β₁₀ on tube formation was investigated as previously described (Lee et al., Cancer Res (2005) 65:137-147).

Briefly, growth factor-reduced Matrigel (200 μl of 10 mg/ml) was added to a 24-well tissue culture plate, followed by polymerization at 37□ for 30 minutes. Uninfected HUVECs (Un) and HUVECs treated with each of Ad, GFP-AdTβ₁₀, and GFP-AdTβ₁₀+siRNA as described in Example 4 were seeded on the surface of the Matrigel at a concentration of 1×10⁵ cells each. Cells were then incubated for 48 hours with or without 10 ng/ml of VEGF in an M199 medium containing 1% FBS. Morphological changes of the cells were observed under the microscope (×400). The length of HUVEC tubes was determined using an inverted microscope equipped with a digital CCD camera (Zeiss) and quantified using ImageLab imaging software (MCM Design). The experiments were carried out in triplicate and each value represents the mean±standard deviation (SD) of the triplicate samples.

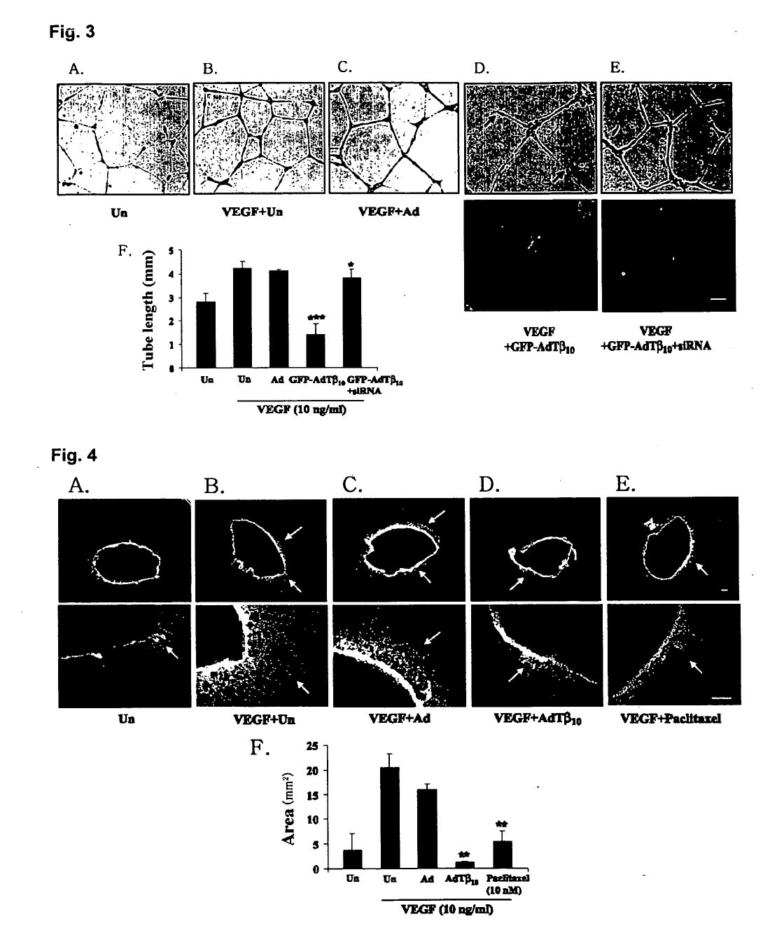

As shown in FIG. 3, an organized network of endothelial cells on Matrigel was formed in response to VEGF stimulation in HUVECs not treated with thymosin β₁₀ (Un+VEGF and Ad+VEGF, FIGS. 3A and 3B, respectively). In contrast, by the expression of thymosin β₁₀ in VEGF-treated HUVECs (GFP-AdTβ₁₀+VEGF, FIG. 3D), the tube formation was significantly repressed (FIG. 3D) to the level of the negative control (Un, FIG. 3A) which was not stimulated by VEGF. However such inhibitory effect of thymosin ⊖₁₀ on tube formation was abrogated after siRNA transfection (GFP-AdTβ₁₀+VEGF+siRNA, FIG. 3E), indicating that the inhibitory effect was indeed attributed to thymosin β₁₀. The result was in accordance with the length of tube formed as shown in FIG. 3F. These results clearly demonstrate that thymosin β₁₀ of the present invention can be used to effectively inhibit tube formation.

### EXAMPLE 5-2

### The Inhibitory Effect of Thymosin β₁₀ on ex vivo Angiogenesis

The inhibitory effect of thymosin β₁₀ on ex vivo angiogenesis was investigated using an ex vivo explant culture of skeletal muscle on Matrigel as described (Jang et al., Molecular Therapy (2004), 9(3): 464-474)

Briefly, 6 week-old BALB/c mice were anesthetized, and the legs were shaved. The tibialis anterior muscle was removed and washed with PBS (phosphate buffered saline) three times. The muscle was then placed in a 24-well plate containing Matrigel, followed by polymerization at 37□ for 30 minutes. An M199 medium containing 1% FBS with or without 10 ng/ml of VEGF was added thereto and the plates were incubated. After 6 days, outgrowth of capillary-like structures was observed, and then a fresh medium containing either 10 nmol/L of paclitaxel or 2×10⁸ pfu (plaque-forming unit) of adenovirus (Ad or AdTβ₁₀) was added. The medium was replaced every other day. After 5 days, the mean area of vascular sprouting was quantified by an optical imaging technique and ImageLab imaging software. The experiment was repeated three times, and the results represent the mean±standard deviation (SD) of the three samples.

As shown in FIG. 4, numerous capillaries were generated in response to VEGF stimulation in muscle not treated with thymosin β₁₀ (Un+VEGF and Ad+VEGF, FIGS. 4B and 4C, respectively). In contrast, by the expression of thymosin β₁₀ (VEGF+AdTβ₁₀, FIG. 4D) in VEGF-stimulated muscles, the capillary formation was significantly repressed (FIG. 3D) to the level of the negative control (Un, FIG. 3A) which was not stimulated by VEGF. Further, the level of inhibition was even greater than that exerted by Paclitaxel (trade mark: Taxol®, Bristol-Myers Squibb Company, New York, N.Y., USA) inhibitor (VEGF+Paclitaxel, FIG. 4E), a well-known inhibitor of angiogenesis. These results clearly demonstrate that thymosin β₁₀ of the present invention can be used to effectively inhibit capillary formation in tissue.

Thus, the inhibitory effect of thymosin β₁₀ on tube formation and capillary generation makes it a promising candidate for a new inhibitor of angiogenesis which can replace conventional angiogenesis inhibitors.

### EXAMPLE 6

### Interaction of Thymosin β₁₀ with Ras

### EXAMPLE 6-1

### Yeast Two Hybrid Analysis

To examine the underlying mechanism of inhibition of angiogenesis, tumor progression and/or metastasis by thymosin β₁₀, a protein interacting with thymosin β₁₀ was investigated by a well-known yeast two hybrid analysis (Fields, S et al., Nature (1989) 340:245-36). Yeast two hybrid analysis is a useful method for determining direct protein-protein interaction, whose principles are explained in Fields, S et al., ibid. In order to find a protein interacting with thymosin β₁₀ of the present invention, two hybrid analysis was performed as described (Lee et al., Cancer Res (2005) 65:137-147, and Grossel, M et al., Nat Biotechnolog (1999)17:1232-1233).

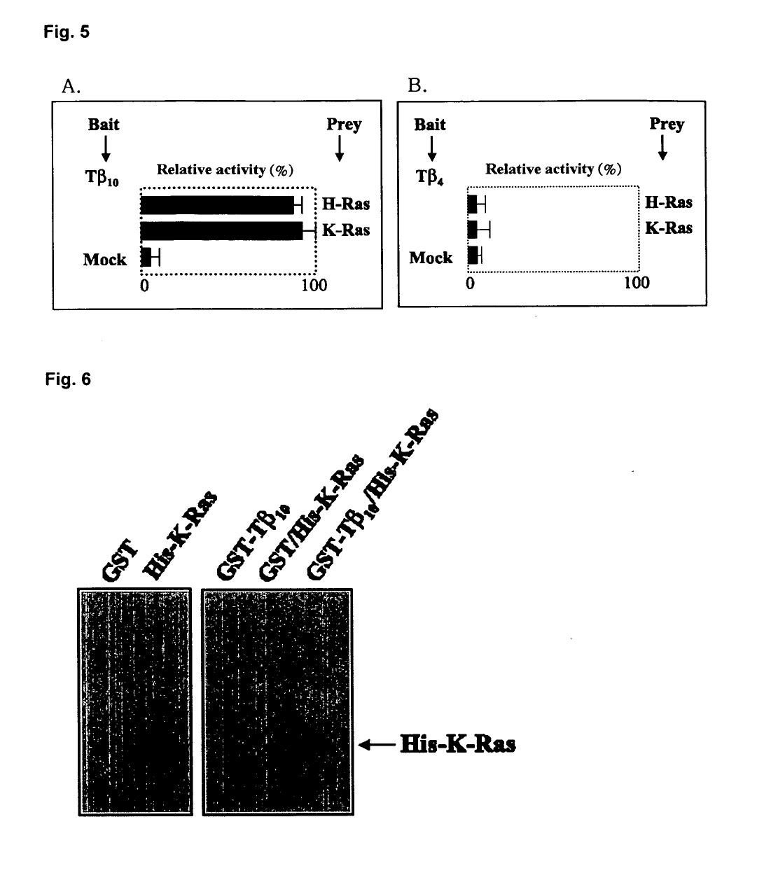

Briefly, for the yeast two hybrid analysis, a first vector that expresses a bait protein (i.e., thymosin β₁₀) fused to the Lex A DNA binding domain and a second vector that expresses a prey protein from cDNA library fused to transactivation domain B42 were used. Lex-A human thymosin β₁₀ or β₄ fusion protein was used to screen the protein that interacts with them and it was found that Ras protein interacts with thymosin β₁₀ but not with β₄. The positive interactions were confirmed by cell growth on a leucine-depleted synthetic medium and blue colony formation on a 5-bromo-4-chloro-3-indolyl-β-D-galactoside (X-gal, 5 mmole/L)-containing medium. The interaction between Ras and thymosin β₄ (FIG. 5B) or β₁₀ (FIG. 5A) was determined by measuring the relative expression level of β-galactosidase in the cells containing thymosin β₄ or β₁₀ and Ras. The activity of β-galactosidase was calculated by the following formula: [l000×(A₄₂₀−1.75×A₅₅₀)]/(reaction time×medium volume×A₆₀₀). A₄₂₀ and A₅₅₀ refer to the absorbance at 420 nm and 550 nm, respectively.

As shown in FIG. 5, not thymosin β₄ (FIG. 5B) but thymosin β₁₀ (FIG. 5A) interacts with Ras, indicated by the high β-galactosidase compared to the negative control (Mock) which does not contain Ras. Thymosin β₄ belongs to the same family as thymosin β₁₀ but does not have anti-angiogenesis activity, thus these results suggest that thymosin β₁₀ exerts its effect through interaction with Ras.

### EXAMPLE 6-2

### Glutathione S-Transferase Pull-Down Assay

To further investigate the interaction of thymosin β₁₀ with Ras, GST-fusion thymosin β₁₀ (GST-Tβ₁₀) and His-fusion K-Ras (His-K-Ras) as prepared in Example 1 were used for a GST pull-down assay as described (Lee et al., Cancer Res (2005) 65:137-147). Briefly, GST-Tβ₁₀ and His-K-Ras were purified by using glutathione sepharose 4B (Amersham Pharmacia Biotech) and Ni-NTA agarose (Qiagen, Chatsworth, Calif., USA), respectively, according to the manufacturer's instructions. Then, an equal amount of GST or GST-T β₁₀ immobilized on gluthathione sepharose beads was incubated with His-K-Ras and washed with PBS to remove unbound His-K-Ras, followed by western blot analysis using anti-His antibody (Oncogene, Uniondale, N.Y., USA) to detect Ras binding to thymosin β₁₀ .

As shown in FIG. 6, Ras protein was detected only in the lane containing both thymosin β₁₀ and Ras (GST-Tβ₁₀/His-K-Ras) but not in the lanes containing either of them, indicating that thymosin β₁₀ interacts directly with Ras.

### EXAMPLE 7

### The Effect of Interaction Between Thymosin β₁₀ and Ras on VEGF-Mediated Ras Signal Transduction Pathway

The interaction between thymosin β₁₀ and Ras suggests that thymosin β₁₀ influences the Ras signal transduction pathway triggered by VEGF by affecting other signal transduction factors, i.e., Raf, Mek, and ERK downstream of Ras, resulting in the inhibition of the Ras signal transduction pathway that leads to angiogenesis, tumor progression, and metastasis. Thus, further experiments were carried out and it was confirmed that interaction between thymosin β₁₀ and Ras resulted in a sequestration of Ras-GTP and/or its interaction with Raf.

### EXAMPLE 7-1

### Thymosin β₁₀ Reduced the Amount of Ras-GTP/Raf Complex

As described in Example 2, Un-, Ad-, or AdTβ₁₀-infected HUVECs were cultured overnight in serum-free media. Then, the cells were stimulated with VEGF (50 ng/ml) for 5 minutes in a phosphate-free M199 medium (Life Technologies) containing 1% FBS. As a negative control, some Un-HUVECs were not treated with VEGF.

The cells were lysed and lysates were prepared according to Meadow K N. et al., J Biol Chem (2001) 276; 49289-49298. The cell lysates were incubated with GST-Raf-RBD harboring only Ras binding domain (RBD), a factor downstream of Ras in the Ras signaling pathway. Ras protein bound to GST-Raf-RBD was isolated by using a Glutathione Disc (Pierce, Rockford, Ill., USA), followed by western blotting using anti-pan-Ras antibody (Clontech, Mountain View, Calif., USA) (Lee et al., Cancer Res (2005) 65:137-147).

The results are shown in FIG. 7. As shown in FIG. 7A, the level of Ras-GTP was increased in cells treated with VEGF (Ad+VEGF), compared with that in cells not treated with VEGF (Un), while the expression of thymosin β₁₀ (AdTβ₁₀+VEGF) significantly reduced the amount of Ras-GTP available for binding to Raf without affecting the total amount of Ras. This result indicates that thymosin β₁₀ binds directly to activated Ras, i.e., Ras-GTP and sequesters Ras-GTP, leading to a decrease in the level of Ras-GTP available for interacting with Raf and thereby reducing the level of Raf/Ras-GTP complex, resulting in inhibition of the Ras signaling pathway.

### EXAMPLE 7-2

### Thymosin β₁₀ Inhibited the Binding of Ras-GTP to Raf

Analysis of Ras bound to GTP or GDP was investigated as previously described (Downward J. et al., Nature (1990) 346; 719-123). Briefly, Un-, Ad-, or AdTβ₁₀ infected HUVECs were cultured overnight in a serum-free medium. The nucleotides in cells were labeled with 0.2 mCi/ml [³²P] orthophosphate in a phosphate-free medium for 3 hours, followed by stimulation with or without (Un) VEGF (50 ng/ml) for 5 minutes. The cells were then lysed and Ras was precipitated from the lysate using an anti-Ras antibody and an IgG antibody as a negative control. Nucleotides were then eluted from the precipitate and analyzed by thin layer chromatography (TLC) using polyethyleneimine-cellulose plates (Sigma). The presence of GTP (Guanosine Triphosphate) and GDP (Guanosine Diphosphate) bound to Ras were assessed by autoradiography (FIG. 7B), and the ratio of GTP to GDP was determined by densitometry (FIG. 7C). The experiment was repeated three times, independently, and consistent results were obtained.

As shown in FIGS. 7B and 7C, the level of Ras-GTP was increased by the treatment of VEGF (VEGF+Ad and VEGF+AdTβ₁₀), compared with the negative control (Un, IgG). In particular, the level of Ras-GTP was more significantly increased by the expression of thymosin β₁₀. This result indicates that thymosin β₁₀ not only reduces the level of Ras-GTP available for binding to Raf through the sequestration of Ras-GTP (Example 7-1), but also inhibits the binding of Ras-GTP to Raf so that it inhibits the conversion of Ras-GTP into Ras-GDP, leading to an increase in the amount of Ras-GTP which, however, may not interact with downstream factors to activate the Ras signaling pathway due to the sequestration by thymosin β₁₀. Thus, through two mechanisms, i.e., direct binding to Ras-GTP or interrupting the binding of Ras-GTP to Raf, thymosin β₁₀ can inhibit the Ras signaling pathway, resulting in the decrease in the amount of Ras-GTP/Raf complexes.

### EXAMPLE 7-3

### Sub-Cellular Localization of Thymosin β₁₀

Sub-cellular localization of thymosin β₁₀ was determined as previously described ) (Lee et al., Cancer Res (2005) 65:137-147; Lee et al., Oncogene (2001) 20:6700-776). Briefly, Un-, Ad- and GFP-AdTβ₁₀ infected HUVECs were stimulated with or without VEGF (50 ng/ml) for 5 minutes. Sub-cellular fractionation of cell lysate, i.e., cytoplasm, cell membrane, and nuclear fractions were prepared, and protein was isolated from each fraction. The presence of thymosin β₁₀, Ras, and alpha-tubulin was determined by western blotting using anti-GFP, anti-Ras (Santa Cruz Biotechnology, Santa Cruz, Calif., USA), and anti-tubulin antibodies (Innogenex, San Ramon, Calif., USA) (FIG. 7D).

As shown in FIG. 7D, thymosin β₁₀ was localized in the cytoplasm as well as the cell membrane, and Ras and alpha-tubulin were found in the cell membrane and the cytoplasm, respectively. This finding indicates that thymosin β₁₀ interacts with Ras in the cell membrane as well as with Raf in the cytoplasm.

### EXAMPLE 8

### Effect of Thymosin β₁₀ on the Activity of Factors Working Downstream of Ras in Ras Signaling Pathway

Experiments were carried out as in Example 7-3 except that whole cell extracts were used and western blotting was performed using anti-GFP, anti-pMEK, anti-pERK, anti-ERK, anti-VEGF (Santa Cruz Biotechnology, Santa Cruz, Calif.), and anti-tubulin antibodies.

Experiments with ovarian cancer cell line 2774 were also carried out as above, except that the secretion of VEGF into media (CCM, concentrated 2774 cell conditioned media) was determined as well as the presence of VEGF in whole cell extraction.

The results are shown in FIGS. 7E to 7G. As shown in FIG. 7E, thymosin β₁₀ inhibited the activation of MEK and ERK, factors working downstream of Ras, by phosphorylation, in HUVEC cells, but did not affect the expression of ERK per se and alpha-tubulin which serves as a positive control. The results indicate that thymosin β₁₀ works by specifically activating the proteins involved in the Ras signaling pathway.

Further, the expression of thymosin β₁₀ inhibited the expression and the secretion of VEGF in endothelial cells (FIG. 7F) and the ovarian cancer cell line (2774) (FIG. 7G).

These results demonstrate that thymosin β₁₀ of the present invention can significantly reduce the expression and secretion of VEGF both in endothelial cells and in tumor cells (see FIGS. 7F and 7G), suggesting that thymosin β₁₀ not only inhibits the autocrine VEGF in endothelial cells but also inhibits paracrine induction of VEGF in tumor cells.

### EXAMPLE 9

### Anticancer Activity of Thymosin β₁₀

### EXAMPLE 9-1

### Establishment of S.C. and Orthotopic Tumor Model

The SPF (specific pathogen free) BALB/c (Biogenomics, Seoul, Korea) and nu/nu mice (Charles River Labs, Wilmington, Mass. USA) were used, and subcutaneous (s.c.) and orthotopic tumor model was establish as previously described (Lee et al., Cancer Res (2005) 65; 137-147).

Briefly, for the s.c. tumor model, l×10⁶ of 2774 tumor cells were hypodermically injected in the mid-dorsal region, and tumors were allowed to grow for 14 days until the average volume reached 100 mm³ before being used for the test described in Example 9-2

For the orthotopic tumor model, 1×10⁶ of GFP-2774 cells were injected into the right ovary through the fat pad. GFP-2774 cells were constructed by a stable transfection of the 2774 cell line with pEGFP-Cl (Clontech) expressing green fluorescent protein (GFP) (Lee et al., Cancer Res (2005) 65; 137-147).

### EXAMPLE 9-2

### Anticancer Activity of Thymosin β₁₀

(1) Anticancer Activity of Thymosin β₁₀ in S.C. Tumor Models

Anticancer activity of thymosin β₁₀ on s.c. tumors was tested as previously described (Lee et al., Cancer Res (2005) 65;137-147). Briefly, the s.c. tumors prepared in Example 9-1 were injected with 1×10⁹ pfu/40 μl of AdTβ₁₀ or Ad once every three days for a total of three times. The tumor volume for each group was then measured every three days with a caliper. On day 27 after the final virus injection, the mice were sacrificed and the tumors were excised and stained with vascular endothelial cell specific anti-mouse CD31 (PECAM-I) antibody (PharMingen, San Diego, Calif., USA) to detect changes of the number of blood vessels by immunohistologic staining. Blood vessel density was calculated by counting the number of blood vessels per cross sections of three different tumors for each group.

The results are shown in FIG. 8. Eight mice were tested for each group and data were presented as the mean±standard deviation (SD) of 8 mice studied. As shown in FIGS. 8A to 8D, tumor volume and the number of blood vessels of mice treated with AdTβ₁₀ was significantly reduced, by 77% and 88%, respectively, compared to that of mice not treated with thymosin β₁₀ (AD). FIG. 8B shows the corresponding tumor volume represented by a bar graph. FIGS. 8C and 8D show a cross-sectional view of the tumor immunohistologically stained with anti-CD13 and the number of blood vessels observed, respectively. These results indicate that thymosin β₁₀ effectively inhibits tumor growth through angiogenesis in vivo.

Furthermore, weight loss or hepatotoxicity was not observed in mice that was treated with thymosin β₁₀.

(2) Anticancer Activity of Thymosin β₁₀ in Orthotopic Tumor Models

Anticancer activity of thymosin β₁₀ on orthotopic tumors was tested as previously described (Lee et al., Cancer Res (2005) 65;137-147). Briefly, in the orthotopic tumor model, a GFP-expressing tumor was examined with an illuminating system (Lightools Research, Encinitas, Calif.). Then, 1×10⁹ pfu/20 μl of AdTβ₁₀ was injected into the tumor and the mouse was sacrificed on day 10 after the injection, and the size of ovarian tumors and the number of blood vessels were measured as in Example 9-1.

The results are shown in FIG. 9. The successful establishment of ovarian tumors was indicated by the green fluorescent region of the abdomen of each mouse upon illumination (FIG. 9A). Eight mice were tested for each group and data were presented as the mean±standard deviation (SD) of 8 mice studied. As shown in FIGS. 9A to 9D, tumor volume and the number of blood vessels of mice treated with AdTβ₁₀ was significantly reduced, by 54% and 77%, respectively, compared to that of mice not treated with thymosin β₁₀ (AD). FIG. 9B shows the corresponding tumor volume represented by a bar graph. FIGS. 9C and 9D show a cross-sectional view of the tumor immunohistologically stained with anti-CD13 and the number of blood vessels observed, respectively. These results indicate that thymosin β₁₀ effectively inhibits tumor growth through angiogenesis in vivo.

All the statistical analyses above were performed by student's t test.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A method of modulating angiogenesis, tumor progression, and/or metastasis comprising the step of administering to a tissue or a subject associated with a disease condition a therapeutically effective amount of thymosin β₁₀ or a nucleotide sequence encoding said protein.

2. The method of claim 1, wherein said modulating inhibits angiogenesis, tumor progression, and/or metastasis.

3. The method of claim 2, wherein said inhibition of angiogenesis, tumor progression, and/or metastasis involves sequestration of Ras-GTP or inhibition of Ras-GTP binding to Raf through direct interaction of thymosin β₁₀ with Ras.

4. The method of claim 3, which further leads to a reduction in the expression of VEGF.

5. The method of claim 4, wherein said condition is a solid tumor.

6. The method of claim 5, wherein said solid tumor comprises colon cancer, ovarian cancer, lung cancer, lymphoma, breast cancer, prostate cancer, and renal cell cancer.

7. The method of claim 6, wherein said administering is conducted in conjunction with chemotherapy.

8. The method of claim 4, wherein said condition is rheumatoid arthritis, psoriasis, diabetic retinopathy, diabetic nephropathy, hypertension, chronic hepatitis, endometriosis, or adiposis.

9. A method of inhibiting Ras activity in a tissue or a subject comprising the step of administering to a tissue or a subject associated with a disease condition a therapeutically effective amount of thymosin β₁₀ or a nucleotide sequence encoding said protein.

10. The method of claim 9, wherein said inhibition of Ras activity involves sequestration of Ras-GTP or inhibition of Ras-GTP binding to Raf through direct interaction of thymosin β₁₀ with Ras.

11. The method of claim 10, which further leads to a reduction in the expression of VEGF.

12. The method of claim 11, wherein said condition is a solid tumor.

13. The method of claim 12, wherein said solid tumor comprises colon cancer, ovarian cancer, lung cancer, lymphoma, breast cancer, prostate cancer, and renal cell cancer.

14. The method of claim 12, wherein said administering is conducted in conjunction with chemotherapy.

15. The method of claim 11, wherein said condition is rheumatoid arthritis, psoriasis, diabetic retinopathy, diabetic nephropathy, hypertension, chronic hepatitis, endometriosis, or adiposis

16. A pharmaceutical composition for modulating angiogenesis, tumor progression, and/or metastasis in a target mammalian tissue comprising thymosin β₁₀ or a nucleotide sequence encoding said protein as an active ingredient, and a pharmaceutically acceptable carrier or excipient.

17. The pharmaceutical composition of claim 16, wherein said modulating inhibits angiogenesis, tumor progression, and/or metastasis.

18. The pharmaceutical composition of claim 16, wherein said target mammalian tissue is associated with a disease condition selected from the group consisting of solid tumors comprising colon cancer, ovarian cancer, lung cancer, lymphoma, breast cancer, prostate cancer, and renal cell cancer; rheumatoid arthritis; psoriasis; diabetic retinopathy; diabetic nephropathy; hypertension; chronic hepatitis; endometriosis; and adiposis.

19. A pharmaceutical composition for inhibiting Ras activity in a target mammalian tissue comprising thymosin β₁₀ or a nucleotide sequence encoding said protein as an active ingredient, and a pharmaceutically acceptable carrier or excipient.

20. The pharmaceutical composition of claim 19, wherein said target mammalian tissue is associated with a disease condition selected from the group consisting of solid tumors comprising colon cancer, ovarian cancer, lung cancer, lymphoma, breast cancer, prostate cancer, and renal cell cancer; rheumatoid arthritis; psoriasis; diabetic retinopathy; diabetic nephropathy; hypertension; chronic hepatitis; endometriosis; and adiposis.
